(19) United States
(12) Patent Application Publication
(10) Pub. No.: US 2022/0176057 A1
(43) Pub. Date: Jun. 9, 2022

(54) METHODS AND MEDICINE DELIVERY DEVICES FOR RESPIRATORY SYSTEM TREATMENT

(71) Applicant: Zewski Corporation, Magnolia, TX (US)

(72) Inventors: Lawrence KILISZEWSKI, The Woodlands, TX (US); Athena Jiunshi CHIEN, San Mateo, CA (US); Rithika Reddy PRODDUTOOR, Frisco, TX (US); Samantha Lee McCLENDON, Marietta, GA (US); Franklin Daniel BRIONES, Kissimmee, FL (US); Hope Kiyomi FA-KAJI, Berkeley, CA (US); Pujita Sai Reddy MUNNANGI, Fremont, CA (US)

(21) Appl. No.: 17/210,163

(22) Filed: Mar. 23, 2021

Related U.S. Application Data
(60) Provisional application No. 63/146,275, filed on Feb. 5, 2021.
(60) Provisional application No. 63/123,352, filed on Dec. 9, 2020.

(51) Int. Cl. A61M 16/06; A61M 16/00; A61M 16/20

(52) U.S. Cl. CPC A61M 16/0672; A61M 16/024; A61M 16/20; A61M 2205/3553; A61M 2230/42; A61M 2230/04; A61M 2230/205; A61M 16/0057

(57) ABSTRACT

The present disclosure relates to methods for treating a respiratory system of a subject, as well as medicine delivery devices for delivering an aerosol medicine to a subject in need thereof. A benefit to the methods herein can be generating a respiratory pattern of a subject that can accurately measure inhalation and exhalation patterns, which can in turn provide a benefit of more efficient and timely delivery of an aerosol medicine to a subject in need thereof. Additional benefits to the methods and devices herein can be helping to improve treatment outcomes, as well as avoiding wastage of expensive medicines. Additional benefits to the medicine delivery devices disclosed herein can be non-invasive, low cost, lightweight, compact, versatile, and simple to use devices useful for a wide range of patients and healthcare settings. Another benefit of the medicine delivery devices can be providing a single-use device that can lower the risk of infection for patients and healthcare providers.

### CROSS-REFERENCE TO RELATED APPLICATIONS

This application claims the benefit of U.S. Provisional Application No. 63/123,352, filed on Dec. 9, 2020, and U.S. Provisional Application No. 63/146,275, filed on Feb. 5, 2021, each of which is incorporated by reference herein in its entirety.

### TECHNICAL FIELD

The present disclosure relates to methods for treating a respiratory system of a subject, as well as medicine delivery devices for delivering an aerosol medicine to a subject in need thereof. A benefit to the methods herein can be generating a respiratory pattern of a subject that can accurately measure inhalation and exhalation patterns, which can in turn provide a benefit of more efficient and timely delivery of an aerosol medicine to a subject in need thereof Additional benefits to the methods and devices herein can be helping to improve treatment outcomes, as well as avoiding wastage of expensive medicines. Additional benefits to the medicine delivery devices disclosed herein can be non-invasive, low cost, lightweight, compact, versatile, and simple to use devices useful for a wide range of patients and healthcare settings. Another benefit of the medicine delivery devices can be providing a single-use device that can lower the risk of infection for patients and healthcare providers.

### BACKGROUND

Respiratory rate is widely used as a sensitive indicator for a range of physiological states. Respiratory rate can be measured directly by a few types of wearable sensors; however, these are typically tight fitting and uncomfortable to wear, or invasive in nature. A much wider range of wearable sensors is available to measure various physiological parameters from a body surface, including sensors that measure electrocardiogram (ECG) and photoplethysmogram (PPG) data. Such sensors can be reasonably comfortable to wear for an extended period of time.

A number of algorithms also exist that can work in connection with the sensors to estimate respiratory rates from the measured physiological data. The available algorithms have been largely designed using data from spontaneously breathing adult subjects, and are primarily limited to measurements of breathing rate. However, the physiological data of subjects receiving respiratory treatment, as well as data from infant subjects, can vary substantially from that of healthy adult patients or patients not undergoing such treatments, which affects the performance of respiratory rate algorithms.

Mechanical ventilation is a widely used respiratory treatment to assist breathing in patients who are not able to breathe properly on their own. Positive pressure breathing assistance systems, such as continuous positive airway pressure (“CPAP”) systems, are conventional for the treatment of respiratory disorders, such as COVID-19, in adults, as well as children and infants. Medicines are frequently delivered during mechanized breathing assistance to patients in need of such treatment for COVID-19, ongoing respiratory distress syndrome (RDS), and other respiratory ailments. Current devices for respiratory treatment and for administering drugs to a patient while receiving assisted breathing treatment are subject to considerable challenges, including low efficiency of drug delivery, high complexity, and high cost. Additional difficulties are presented by the invasive nature of many of the current devices for respiratory treatment, adding to patient discomfort and risk of infection. Drug delivery can also require interruption of respiratory treatment, again adding to patient discomfort and health risks. The current algorithmic methods for respiratory treatment are subject to challenges of accuracy in estimating respiratory rate in subjects undergoing respiratory treatment, including infant subjects. There remains a need for methods of respiratory system treatment and medicine delivery devices that can address these challenges.

### SUMMARY

Embodiments of methods of treating a respiratory system of a subject are disclosed herein. In an embodiment, such a method includes: providing at least one non-invasive sensor; attaching the least one non-invasive sensor to at least one body surface of the subject and configuring the at least one non-invasive sensor to send a sensor signal to a controller; collecting sensor signal data from the at least one non-invasive sensor over a measurement time period; extracting respiratory data from the sensor signal data by applying a bandpass filter; determining an inhalation period and exhalation period of the subject; and actuating a treatment during the inhalation period by sending an actuator signal from the controller to an air pump or a medicine delivery device, wherein the pump or medicine delivery device is connected by a breathing apparatus connected to the respiratory system of the subject.

In certain embodiments, the method further includes generating a respiratory pattern of the subject by applying an algorithm to the extracted respiratory data, wherein applying the algorithm comprises calculating derivatives of the extracted respiratory data as a function of time to form a derivative curve. In certain embodiments, an inflection point of the extracted respiratory data corresponding to a change in sign of the derivative curve corresponds to a time of onset of an inhalation period or a time of onset of an exhalation period.

In certain embodiments, the measurement time period is from about 10 seconds to about 2 minutes; or wherein the measurement time period comprises from about 3 to about 120 repeated inhalation periods or exhalation periods of the subject.

In certain embodiments, applying the bandpass filter includes applying a lower cutoff inhalation or exhalation frequency of about 0.33 Hz and a higher cutoff inhalation or exhalation frequency of about 1 Hz; or wherein applying the bandpass filter includes applying a sensor signal data sampling frequency of about 250 Hz.

In certain embodiments, the subject is a human, an infant, an unconscious patient, a patient receiving a mechanically assisted breathing treatment, a ventilated patient, a cat, a dog, a horse, or a mammal.

In some embodiments of methods herein, the at least one non-invasive sensor comprises an electrocardiogram (ECG) sensor, wherein attaching the least one non-invasive sensor comprises attaching at least one ECG lead to the body surface of the subject, and the sensor signal data comprises ECG signals. In certain embodiments, the method includes attaching at least two ECG leads to the body surface of the subject, wherein the body surface includes a chest surface, an arm surface, a leg surface, or a combination thereof; and the sensor signal data comprises ECG signals collected from the at least two ECG leads.

In some embodiments of methods herein, the at least one non-invasive sensor comprises a pulse oximeter sensor, and the body surface includes a finger surface, a toe surface, an ear surface, or a combination thereof; and wherein the sensor signal data includes oxygen saturation level data.

Embodiments of methods of delivering an aerosol medicine to a subject in need thereof are disclosed herein. In an embodiment, such a method includes: providing a medicine delivery device, wherein the medicine delivery device comprises an aerosol medicine dispenser connected by an air flow system to an actuator, and a programmable control module configured to control the actuator and the aerosol medicine dispenser; providing at least one non-invasive sensor; attaching the at least one non-invasive sensor to at least one body surface of the subject and configuring the at least one non-invasive sensor to send a sensor signal to the programmable control module; collecting sensor signal data from the at least one non-invasive sensor over a measurement time period; extracting respiratory data from the sensor signal data by applying a bandpass filter; determining an inhalation period and an exhalation period of the subject; and actuating a treatment during the inhalation period by sending an actuator signal from the programmable control module to the actuator or the aerosol medicine dispenser, wherein the pressure valve or aerosol medicine dispenser is connected by a breathing apparatus connected to the respiratory system of the subject.

In certain embodiments, the method further includes programming the programmable control module to dispense an amount of medicine for a treatment frequency during a treatment duration. In certain embodiments, the method includes programming the programmable control module to dispense an amount of medicine once per a number of inhalation periods.

In some embodiments, the actuator comprises a pressure valve. In such embodiments, the method further includes connecting the pressure valve to a pressure source. In certain embodiments, the method further includes flowing a treatment volume of medicine from the aerosol medicine dispenser into the air flow system during an inhalation period. In some embodiments, the method optionally includes closing the pressure valve during an exhalation period.

In certain embodiments, the at least one non-invasive sensor includes an electrocardiogram (ECG) sensor, wherein the method includes attaching the least one non-invasive sensor comprises attaching at least one ECG lead to the body surface of the subject, and the sensor signal data comprises ECG signals. In certain embodiments, the method includes attaching at least two ECG leads to the body surface of the subject, wherein the body surface includes a chest surface, an arm surface, a leg surface, or a combination thereof; and the sensor signal data comprises ECG signals collected from the at least two ECG leads.

In certain embodiments, the at least one non-invasive sensor comprises a pulse oximeter sensor, and the body surface includes a finger surface, a toe surface, an ear surface, or a combination thereof; and wherein the sensor signal data includes oxygen saturation level data.

Embodiments of a medicine delivery device are disclosed herein. In various embodiments, the medicine delivery device includes: an aerosol medicine dispenser connected by an air flow system to an actuator; at least one non-invasive sensor configured to attach to at least one body surface of a subject; and a programmable control module configured to receive sensor signals from the at least one non-invasive sensor and configured to control the actuator and the aerosol medicine dispenser.

In certain embodiments, the programmable control module comprises machine-readable code configured to: collect sensor signal data from the at least one non-invasive sensor over a measurement time period; extract respiratory data from the sensor signal data by applying a bandpass filter; determine an inhalation period and exhalation period of the subject; and actuate a treatment during the inhalation period by sending an actuator signal from the programmable control module to the actuator or the aerosol medicine dispenser.

In certain embodiments, the at least one non-invasive sensor comprises an electrocardiogram (ECG) sensor and one or more ECG leads. In some embodiments, the at least one non-invasive sensor includes a pulse oximeter.

In certain embodiments, the aerosol medicine dispenser includes a medicine delivery controller connected to a dispensing opening of a medicine reservoir, wherein the medicine delivery controller is connected to the air flow system, and wherein the medicine delivery controller comprises a nebulizer, an aerosolizer, an atomizer, a pressurized metered dose inhaler, a vaporizer, a fan, a hopper, a dry powder inhaler, a diffuser, a vibrating piezoelectric aerosolizer, or a combination thereof. In certain embodiments, the actuator comprises a pressure valve, a flexible bellows, a motor, a hand pump, a solenoid valve, an air flow valve, or a combination thereof.

In certain embodiments, the medicine delivery device further includes a subject interface configured to connect to the air flow system, wherein the subject interface includes a nasal cannula, a face mask, a breathing tube, a medicine port, or a combination thereof.

In certain embodiments, the air flow system includes at least one air tube, at least one air pipe, at least one air path, or a combination thereof; or wherein the actuator is configured to connect to at least one pressure source, and optionally, the at least one pressure source comprises an air pump, an air tank, an air tube, an air line, or a combination thereof.

In certain embodiments, the medicine delivery device further includes at least one electrical connection, wherein the at least one electrical connection connects the programmable control module to the at least one non-invasive sensor, the programmable control module to the actuator, the programmable control module to the aerosol medicine dispenser, or a combination thereof. In certain embodiments, at least one of the programmable control module, the at least one non-invasive sensor, the actuator, and the aerosol medicine dispenser comprises a wireless transmitter, a wireless receiver, or a combination thereof.

### BRIEF DESCRIPTION OF THE DRAWINGS

The foregoing summary, as well as the following detailed description of the embodiments, will be better understood when read in conjunction with the attached drawings. For the purpose of illustration, there are shown in the drawings some embodiments, which may be preferable. It should be understood that the embodiments depicted are not limited to the precise details shown. Unless otherwise noted, the drawings are not to scale.

FIG. 1 shows a flow chart illustrating a method of treating a respiratory system of a subject, according to embodiments disclosed herein.

FIG. 2 shows a graph of original and filtered neonatal ECG data with time, according to embodiments disclosed herein.

FIG. 3 shows a graph of ECG derived respiratory data (ECGDR) over time, according to embodiments disclosed herein.

FIG. 4A shows a graph of neonatal respiratory monitor rate (RMR) data with time, according to embodiments disclosed herein.

FIG. 4B shows a graph of neonatal respiratory monitor rate (RMR) data with time, according to embodiments disclosed herein.

FIG. 5A shows a graph of infant respiratory monitor data (RMR) and ECGDR over time, according to embodiments disclosed herein.

FIG. 5B shows a histogram of differences between infant inhalation start times as measured by RMR versus ECGDR data, according to embodiments disclosed herein.

FIG. 5C shows a distribution graph of inhalation times for different inhalation durations as measured by infant RMR data, according to embodiments disclosed herein.

FIG. 5D shows a distributions graph of inhalation times for different inhalation durations as measured by infant ECGDR data, according to embodiments disclosed herein.

FIG. 5E shows a histogram of distributions of inhalation durations times for infant RMR derived respiratory data and infant ECGDR data, according to embodiments disclosed herein.

FIG. 6 shows a graph of simultaneously collected ECG data and measured inhalation duration data over time, according to embodiments disclosed herein.

FIG. 7A shows a graph of ECG derived respiratory data (ECGDR) and measured inhalation duration data over time, according to embodiments disclosed herein.

FIG. 7B shows a dot plot of inhalation detection differences derived from ECGDR data and corresponding respiratory data per number of inhalations, according to embodiments disclosed herein.

FIG. 7C shows a histogram of inhalation detection differences derived from ECGDR data and corresponding respiratory data at different times, according to embodiments disclosed herein.

FIG. 8 shows a flow chart illustrating a method of generating a respiratory pattern of a subject by applying an algorithm to extracted respiratory data, according to embodiments of methods disclosed herein.

FIG. 9 shows a flow chart illustrating a method of generating a respiratory pattern of a subject by applying an algorithm to extracted respiratory data, according to embodiments of methods disclosed herein.

### DETAILED DESCRIPTION

Unless otherwise noted, all measurements are in standard metric units.

Unless otherwise noted, all instances of the words “a,” “an,” or “the” can refer to one or more than one of the word that they modify.

Unless otherwise noted, the phrase “at least one of” means one or more than one of an object. For example, “at least one of a single walled carbon nanotube, a double walled carbon nanotube, and a triple walled carbon nanotube” means a single walled carbon nanotube, a double walled carbon nanotube, or a triple walled carbon nanotube, or any combination thereof.

Unless otherwise noted, the term “about” refers to ±10% of the non-percentage number that is described, rounded to the nearest whole integer. For example, about 250 Hz, would include 225 to 275 Hz. Unless otherwise noted, the term “about” refers to ±5% of a percentage number. For example, about 20% would include 15 to 25%. When the term “about” is discussed in terms of a range, then the term refers to the appropriate amount less than the lower limit and more than the upper limit. For example, from about 10 seconds to about 2 minutes would include from 9.5 seconds to 2.1 minutes.

Unless otherwise noted, properties (height, width, length, ratio etc.) as described herein are understood to be averaged measurements.

Unless otherwise noted, the terms “provide”, “provided” or “providing” refer to the supply, production, purchase, manufacture, assembly, formation, selection, configuration, conversion, introduction, addition, or incorporation of any element, amount, component, reagent, quantity, measurement, or analysis of any method or system of any embodiment herein.

Unless otherwise noted, the term “aerosol” refers to a suspension of liquid or solid particles in air or a gas.

Unless otherwise noted, the term “medicine” refers to a drug, medicine, aerosol medicine, medicament or therapeutic agent.

Unless otherwise noted, the term “subject” refers to a patient, a human, an infant, or an animal, including mammals.

Unless otherwise noted, the term “trough” refers to a derivative of extracted respiratory data changing from less than zero to greater than zero over a time period.

Respiration monitoring is an important technique for treating and managing a wide variety of conditions, including stress and sleep disorders. Sensors that directly monitor respiratory rate can be used, but these require the use of invasive equipment such as thermistors, spirometers, or respiratory belts that are uncomfortable to wear, particularly for prolonged periods. Such sensors are not only uncomfortable, but expensive. Since it has been shown that electrocardiogram (ECG) and pulse oximetry (photoplethysmogram or PPG) signals can be used to approximate respiratory rate as well as respiratory wave morphology, it has been possible to use a wide range of wearable sensors, such as ECG sensors and pulse oximetry sensors, to monitor respiration. These types of sensors are generally far more comfortable to wear, as well as inexpensive compared to the direct respiration measurement techniques. A number of algorithms also exist that can estimate respiratory rate from ECG and PPG data. The use of ECG and PPG sensors can accordingly be advantageous for use in respiratory monitoring.

Patients suffering from a respiratory disorder may require ongoing respiratory monitoring in order to manage their condition, and may need a mechanically assisted breathing treatment for a period of time. Patients undergoing treatment for respiratory disorders are frequently administered medicines while undergoing assisted breathing treatment, commonly during CPAP or oxygen supplementation. Many patients, including adults as well as children and infants, need additional drugs for treating ongoing respiratory distress syndrome (RDS). RDS is caused by many underlying problems, among them premature birth, pneumonia, and other respiratory diseases, including severe acute respiratory syndromes, most recently COVID-19.

Mechanically assisted breathing action can be accomplished using a conventional form of ventilation treatment typically used for respiratory disorders, including a positive pressure breathing system or ventilator, such as a continuous positive airway pressure (CPAP) system, a nasal CPAP (NCPAP) system, or a bilevel positive airway pressure (BiPaP) system. Such systems use a constant positive pressure from an air source, or a gas source containing oxygen, to keep the airways dilated during inhalation and supply the patient with oxygen. The air or gas is pressurized and supplied from the air source or gas source through an air flow system. The air flow system can be connected to a subject interface, which commonly includes nasal prongs or a nasal cannula, nasopharyngeal tubes, a face mask, or an endotracheal tube. Positive pressure ventilator systems maintain a continuous positive airway pressure by using a restrictive air outlet device, or a pressure valve. The pressure valve can be located before, at the patient interface, or beyond the patient interface in the airflow path. While the machine is in operation, the pressure valve allows air to flow normally through the airflow path, but when pressure is eliminated, the valve seals to prevent backflow, thus preventing moisture or oxygen from flowing into the machine from the airflow path.

Administering drugs while a patient is on a positive pressure ventilator machine can be challenging. The current devices that allow drug delivery during treatment are plagued by low efficiency in drug delivery to the patient. Data from current systems show in many cases that less than 15% of the dosed drug actually makes it to the patient's lungs. This low efficiency is due in part to pressure-assisted systems utilizing a constant positive pressure to keep the airways dilated and prevent their collapse. When medicines are administered by being pumped into the lungs, the constant positive pressure results in the medicine being pumped continuously, while the patient is exhaling as well as inhaling. This results in some of the medicine being blown out of a pressure valve in the system, thus wasting some of the drug. In the case of liquid medications that are pumped into the lungs, the continuous positive pressure can result in liquid building up in the lungs. Treatment of infants with liquid medications using intubation, such as treatment with liquid surfactants, can result in the liquid pooling in the lungs. Not only do these caveats present difficulties for the effective treatment of patients, but many of the drugs used in these treatments are quite expensive, thus greatly increasing costs from the need to use considerably more of the drug to achieve the desired dosage. These medicines are in liquid form which makes them more expensive to store, ship, and purchase.

Systems that time drug delivery to coincide with patient inhalation have been developed; such systems have been shown to be able to increase drug delivery to 40% or more of the dosed drug to the lungs. While helping to solve the problems with efficiency, however, the current devices that allow for delivery of drugs during pressure-assisted breathing treatment are also quite expensive. They are also large, cumbersome, and difficult to transport. Many such devices are also not all inclusive, meaning that several products must be separately purchased in order to achieve the patient treatment goals.

Currently available devices are also typically designed to be cleaned and re-used between patients. One reason for such re-use is the high cost of such devices. The re-use of these devices, as well as the necessity to remove and connect or re-connect several different parts of the system, increases the risk of infection for the patients and healthcare providers. Preventing infection transmission has always been a challenge in healthcare settings, but it is of even greater importance in the present day of global pandemics.

Currently available algorithms have generally been developed using ECG or pulse oximetry data collected from healthy adult subjects. Healthy adults tend to have regular breathing patterns, thus providing a more uniform influence on the ECG or PPG data from which the respiratory rate or respiratory wave patterns are derived. In the case of patients receiving respiratory treatment such as mechanically assisted breathing, however, the treatment is likely to affect the physiology of ECG and PPG respiratory modulations. Algorithms reflecting healthy adult breathing patterns generally measure breathing rate only and may therefore not lend themselves to the greatest accuracy in analyzing with specificity the times of onset or durations of respiratory patterns of a subject receiving an assisted breathing treatment. Likewise, algorithms derived using data collected from adults may not present the most accurate measurements of the times of onset and durations in a breathing pattern of an infant.

There remains a need for methods of treating a respiratory system of a subject that can provide greater precision and accuracy in the timing of delivery of a treatment to coincide with the desired inhalation period of a patient during respiratory treatment, for adult as well as infant patients. There remains a need for medicine delivery devices that can not only efficiently and safely administer drugs to a patient during treatment with a positive pressure ventilator, but that can provide low cost devices that are lightweight, compact in size, and simple to use.

The methods of the present disclosure can provide such precision and accuracy that in turn can allow the efficient and safe delivery of a treatment at or during a desired inhalation period for an adult or infant patient during a respiratory system treatment. The medicine delivery devices of the present disclosure include algorithms that can allow more accurate monitoring of patient breathing patterns. Such devices can be programmed to dispense a medicine more accurately and efficiently during patient inhalation, and withhold the medicine during patient exhalation in the course of a respiratory treatment. This design can avoid wasting medicine, avoid clogging the lines with medicine, and avoid inadvertently filling the patient's lungs with liquid or solid particles.

The medicine delivery devices of the present disclosure can be disposable, single use, or for single-patient use to reduce the risk of contaminating patients or medical staff, and avoids the need for costly and/or time consuming cleaning of the medical device between patients. In some embodiments, medicine delivery devices can also provide advantages of effective low cost reusable alternatives.

The medicine delivery devices of the present disclosure can enable the delivery of inexpensive, easily stored and transported solid medicines, which can then be dispensed as a solid particle in a gas or dissolved in a liquid for dispensing as liquid droplet in a gas. The medicine delivery devices of the present disclosure can provide an advantageous design that is modular and makes use of low cost, commercially available components for providing greater access to medicine delivery systems in poor or remote areas. Embodiments of respiratory treatment methods and medicine delivery devices herein can provide an important benefit of suitability for nearly any patient in any healthcare setting, including patients receiving a respiratory treatment and infant patients, thus helping to reduce recovery times and improve patient outcomes.

### Embodiments of Methods of Treating a Respiratory System

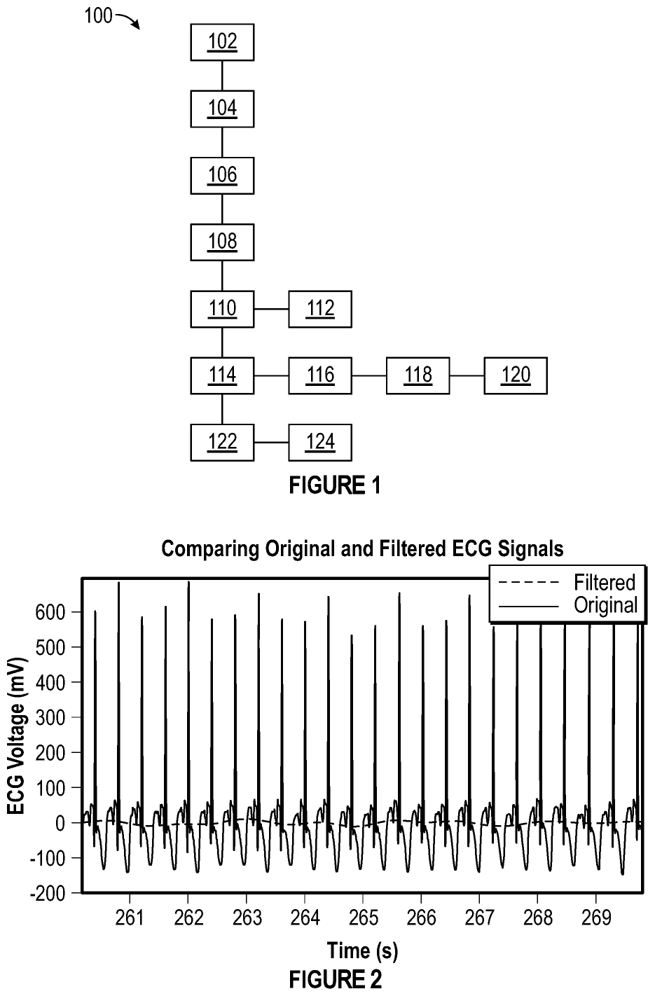

Embodiments of methods of treating a respiratory system of a subject are disclosed herein. As an embodiment of a method disclosed herein, referring to FIG. 1, the method 100 includes: providing at least one non-invasive sensor 102; attaching the at least one non-invasive sensor to at least one body surface of the subject 104, and configuring the at least one non-invasive sensor to send a sensor signal to a controller 106; collecting sensor signal data from the at least one non-invasive sensor over a measurement time period 108; extracting respiratory data from the sensor signal data 110 by applying a bandpass filter 112; determining an inhalation period and exhalation period of the subject 114 by calculating a derivative of filtered sensor signal data 116, detecting at least one trough in the calculated derivatives 118, wherein a derivative at a current filtered sensor signal data point is greater than zero, or a derivative at a previous filtered sensor signal data point is less than zero, and storing a current time point in a data array 120; and actuating a treatment during the inhalation period 122 by sending an actuator signal from the controller to an air pump or a medicine delivery device 124, wherein the pump or medicine delivery device is connected by a breathing apparatus connected to the respiratory system of the subject.

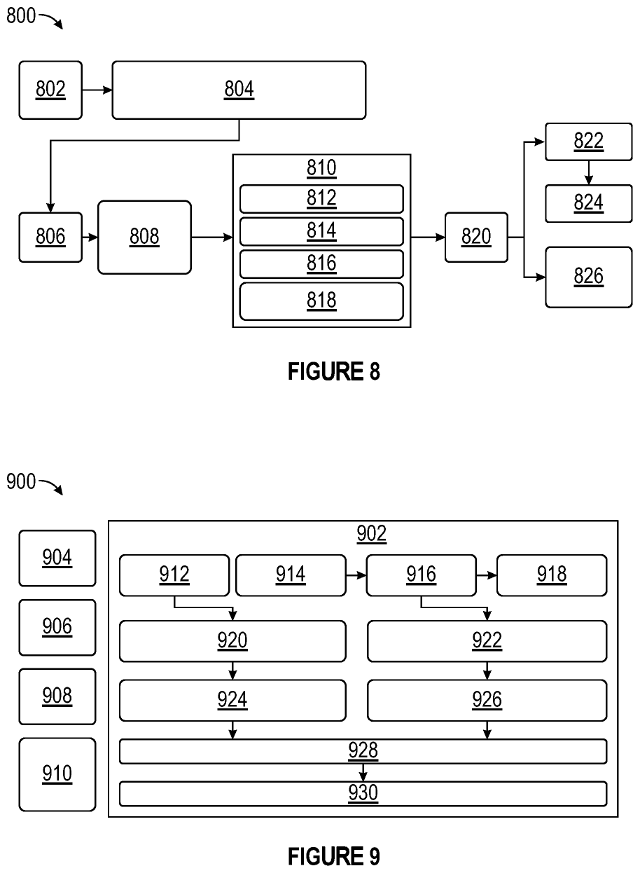

Various embodiments of methods disclosed herein include generating a respiratory pattern of a subject by applying an algorithm to extracted respiratory data. As an embodiment of a method disclosed herein, referring to FIG. 8, the method 800 includes: include bandpass filters library 802; initialize variables for bandpass filter cutoff frequencies and derivative calculations 804; begin timer 806; create bandpass filters 808; execute while loop 810, wherein while loop 810 includes: collect sensor signal data 812, apply bandpass filter to extract respiratory data 814, calculate derivatives to determine an inhalation period or an exhalation period 816, calculate delay after start of inhalation to the time inhalation is detected 818; end timer 820; save data into .csv 822; visualize data in a programming language 824; and instantly plot data in computing platform 826. As an embodiment of a method disclosed herein and a further illustration of while loop 810 in FIG. 8, referring to FIG. 9, the method 900 includes: while loop 902, wherein while loop 902 includes: collect data 904, apply bandpass filter 906, calculate derivatives 908, and detect inhalation and calculate delay 910; wherein collect data 904 includes: collect button pressing and display 912 and collect ECG trace 914; apply bandpass filter 906 includes: filter ECG trace and display 916 and store time of filtered data point and display 918; wherein calculate derivatives 908 includes: calculate derivative of button pressing 920 and calculate derivative of filtered ECG 922; wherein detect inhalation and calculate delay 910 includes: use button derivative at current point to determine start of inhalation (positive edge) 924; use ECG derivatives at current point and previous point to determine start of inhalation (peak) 926; print values, store time, and calculate and display delay 928; and set ECG derivative at previous point to be the current derivative for the next iteration 930.

Embodiments of a method of treating a respiratory system of a subject are disclosed herein. In various embodiments, the method includes providing at least one non-invasive sensor;

attaching the least one non-invasive sensor to at least one body surface of the subject and configuring the at least one non-invasive sensor to send a sensor signal to a controller;

collecting sensor signal data from the at least one non-invasive sensor over a measurement time period; extracting respiratory data from the sensor signal data by applying a bandpass filter;

determining an inhalation period and exhalation period of the subject; and actuating a treatment during the inhalation period by sending an actuator signal from the controller to an air pump or a medicine delivery device, wherein the pump or medicine delivery device is connected by a breathing apparatus connected to the respiratory system of the subject.

In some embodiments of methods herein, the at least one non-invasive sensor includes an electrocardiogram (ECG) sensor. Such embodiments can provide a benefit of a sensor that is comfortable to wear by the subject, even for an extended period of time. Use of a non-invasive sensor such as an ECG sensor contrasts to wearable sensors that are available to monitor respiratory rate directly, which are invasive or uncomfortable to wear. In various embodiments, attaching the least one non-invasive sensor includes attaching at least one ECG lead to a body surface of a subject. In some embodiments, attaching the sensor can include pre-cleaning the body surface of the subject or the sensor, or applying a conductive gel, pre-adhesive or polymer to the body surface before attaching the sensor. In some embodiments, the sensor can include an adhesive layer to help the sensor adhere in place to the body surface. The body surface can include a skin surface, a chest surface, an arm surface, a leg surface, or a combination thereof. Such embodiments can provide a benefit of increasing the versatility of the method for use with a variety of different subjects and in various healthcare settings. In certain embodiments, the subject is a human, an infant, an unconscious patient, a patient receiving a mechanically assisted breathing treatment, a ventilated patient, a cat, a dog, a horse, or a mammal. In some embodiments, one or more ECG leads can be attached to a chest surface of an infant.

In various embodiments, the respiratory treatment method includes a mechanically assisted breathing treatment; such a treatment can include a conventional form of ventilation treatment typically used for respiratory disorders, including a positive pressure breathing assistance or ventilator system, such as a continuous positive airway pressure (CPAP) system, a nasal CPAP (NCPAP) system, or a bilateral positive airway pressure (BiPap) system. In certain embodiments, the respiratory treatment method includes a stress related treatment or a sleep disorder treatment, such as a sleep apnea treatment.

In certain embodiments, the method includes attaching at least one ECG lead to the body surface of the subject. In such embodiments, the sensor signal data includes ECG signals from the at least one ECG lead. In certain embodiments, the method includes attaching at least two ECG leads to the body surface of the subject, wherein the body surface includes a chest surface, an arm surface, a leg surface, or a combination thereof. In such embodiments, the sensor signal data includes ECG signals collected from the at least two ECG leads. In an embodiment, three ECG leads are attached to a body surface of the subject; in some embodiments, the three ECG leads are attached to a left arm surface, a right arm surface, and either a right leg surface or a left leg surface of the subject. In such embodiments, the sensor signal data includes ECG signals from the three ECG leads. Such embodiments can provide benefits of increasing the quantity of sensor signals that can be collected from the subject per measurement time period, which can increase the accuracy and efficiency of extracting respiratory data from the sensor signal data, and in turn increase the accuracy and efficiency of respiratory system treatments. Such embodiments can provide benefits of a versatile, accurate, efficient, non-invasive and comfortable method to provide a respiratory system treatment, including such a treatment for a subject in need of a mechanically assisted breathing treatment. Such embodiments can also provide a benefit of continuous wear of the device throughout respiratory treatment, thus avoiding any need to interrupt wearing of the device to administer a respiratory treatment.

In some embodiments of methods herein, the at least one non-invasive sensor includes a pulse oximeter sensor, and the body surface includes a finger surface, a toe surface, an ear surface, or a combination thereof. In such embodiments, the sensor signal data includes oxygen saturation level data. In some embodiments, the at least one non-invasive sensor can include an ECG sensor and a pulse oximeter sensor; in such embodiments, the sensor signal data includes ECG signals and oxygen saturation level data. Such embodiments can provide a benefit of greater versatility for the embodied methods of respiratory system treatment for different subjects and in various healthcare situations.

In certain embodiments, the method further includes generating a respiratory pattern of the subject by applying an algorithm to the extracted respiratory data, wherein applying the algorithm comprises calculating derivatives of the extracted respiratory data as a function of time to form a derivative curve. In certain embodiments, an inflection point of the extracted respiratory data corresponding to a change in sign of the derivative curve corresponds to a time of onset of an inhalation period or a time of onset of an exhalation period. Such embodiments can provide a benefit of greater accuracy in respiratory treatments, by allowing for the actuation of a treatment at the time of onset of an inhalation period, or more accurately during an inhalation period, or a combination thereof.

In certain embodiments, the measurement time period is from about 10 seconds to about 2 minutes. In certain embodiments, the measurement time period includes from about 3 to about 120 repeated inhalation periods or exhalation periods of the subject. In certain embodiments, the measurement time period includes from about 10 to about 100 repeated inhalation periods or exhalation periods of the subject. In certain embodiments, the measurement time period includes from about 30 to about 60 repeated inhalation periods or exhalation periods of the subject.

In certain embodiments, applying the bandpass filter includes applying a lower cutoff inhalation or exhalation frequency of about 0.33 Hz and a higher cutoff inhalation or exhalation frequency of about 1 Hz. In certain embodiments, applying the bandpass filter includes applying a lower cutoff inhalation or exhalation frequency of about 0.4 Hz and a higher cutoff inhalation or exhalation frequency of about 0.9 Hz. In certain embodiments, applying the bandpass filter includes applying a lower cutoff inhalation or exhalation frequency of about 0.6 Hz and a higher cutoff inhalation or exhalation frequency of about 0.8 Hz. In certain embodiments, applying the bandpass filter includes applying a sensor signal data sampling frequency of about 250 Hz.

### Embodiments of Medicine Delivery Devices

Embodiments of a medicine delivery device are disclosed herein. In various embodiments, the medicine delivery device includes: an aerosol medicine dispenser connected by an air flow system to an actuator at least one non-invasive sensor configured to attach to at least one body surface of a subject; and a programmable control module configured to receive sensor signals from the at least one non-invasive sensor and configured to control the actuator and the aerosol medicine dispenser. In certain embodiments, the programmable control module is configured to directly control the aerosol medicine dispenser by triggering the dispenser to turn on to dispense an aerosol medicine at a desired time, or to turn off to stop dispensing an aerosol medicine at a desired time.

In certain embodiments, the aerosol medicine dispenser includes a medicine delivery controller connected to a dispensing opening of a medicine reservoir, wherein the medicine delivery controller is connected to the air flow system, and wherein the medicine delivery controller comprises a nebulizer, an aerosolizer, an atomizer, a pressurized metered dose inhaler, a vaporizer, a fan, a hopper, a dry powder inhaler, a diffuser, a vibrating piezoelectric aerosolizer, or a combination thereof. In certain embodiments, the aerosol medicine dispenser is disposable.

In certain embodiments, the actuator includes a pressure valve, a flexible bellows, a motor, a hand pump, a solenoid valve, an air flow valve, or a combination thereof. In certain embodiments wherein the actuator includes a flexible bellows, the aerosol medicine dispenser includes a check valve, a one-directional flow valve, a no-flow valve, or a combination thereof. Such embodiments can provide a benefit of preventing backflow of an aerosol medicine.

In certain embodiments, the medicine delivery controller includes an actuator, such as a bellows, connected to an ultrasonic atomizer. In such embodiments, the ultrasonic atomizer can include an atomizer disc or fogger. In certain embodiments, the atomizer is connected to a power source by one or more electrical connections. Such a power source can include one or more switches, such as an on/off switch, to control the atomization disc. In some embodiments, the power source can be connected to a programmable control module or printed circuit board (PCB). In certain embodiments, the power source can be programmed to control the switch connected to the atomization disc, to turn the disc on or off, or to control an amount or rate of voltage supplied to the atomization disc. Such embodiments can provide benefits of controlling timing or the amount or rate of delivery of medicine to the subject through the air flow system, by controlling the action of the atomizer. In certain embodiments, an aerosol flow path is connected to the atomizer, wherein the aerosol flow path is connected to an air flow system and a subject interface. In certain embodiments, the aerosol flow path can be connected to a subject interface connector. Such a subject interface connector can include a plastic or rubber material. In certain embodiments, the aerosol flow path can include an air intake port. In certain embodiments, the air intake port can be connected to a solenoid valve that can be used to control the flow of air through the air intake port.

In certain embodiments, the medicine delivery controller includes a medicine reservoir connected to an ultrasonic atomizer, and includes a bellows connected between the ultrasonic atomizer and an aerosol flow path. In certain embodiments, the action of the bellows can be controlled by a bellows controller connected to the bellows. In such embodiments, the bellows controller can include one or more compression arms connected to a top or a bottom portion of the bellows, the one or more compression arms being connected to a vertical rack. In certain embodiments, the one or more compression arms is controlled by one or more gears connected to the vertical rack. In certain embodiments, the one or more gears includes a motor driven gear; in such embodiments, the one or more gears can be connected to a motor shaft to allow a connected gear to rotate in a clockwise or counterclockwise direction. In certain embodiments of a motor driven gear, the gear can include a one-way bearing having an outer rotor, an inner rotor, and rollers to control the rotation of the gear in either direction. In certain embodiments, such a gear can rotate in a free flow direction but not rotate in the opposite or “no spin” direction. In such embodiments, in the no-spin direction, rollers move up the vertical rack and stop rotation via friction with the outer rotor. In such embodiments, the “no spin” direction can result in compression of the bellows, because the gear does not rotate along the vertical rack, while the motor shaft rotates. In the free flow direction, the gear rotates around a stationary motor shaft and moves along the vertical rack, resulting in expansion of the bellows. In such embodiments, the bellows actuator allows a unidirectional activation of the bellows. In certain embodiments, the free flow rotation is in a counterclockwise direction, and the no-spin direction is clockwise. In some embodiments, the bellows actuator can include a solenoid, worm-gear, or piston driven actuator.

In certain embodiments, the medicine delivery controller includes a bellows connected to an aerosol flow path, a water reservoir, and a bellows controller. In certain embodiments, the medicine delivery controller includes a bellows that also serves as a medicine reservoir. In certain embodiments, the bellows includes flexible walls. Such flexible walls can include a plastic or a rubber material. In such embodiments, the bellows contains a medicine; in certain embodiments, the medicine is located in a bottom portion of the bellows, or adjacent to a flexible bottom wall of the bellows. In certain embodiments, the bellows can be pre-filled with a medicine, such as a liquid medicine, or filled with a medicine by an operator. In certain embodiments, an ultrasonic atomizer can be connected to or located in proximity to a portion of the bellows containing the medicine, such as a flexible bottom wall; in such embodiments, a medicine aerosol can be generated in the bellows by sonication or vibration of the atomization disc. Such embodiments can provide a benefit of allowing condensation generated on an inner surface of the bellows to be contained within the bellows by falling or draining back into the interior of the bellows. Such embodiments can provide benefits of avoiding waste of valuable drugs, as well as greater precision in medicine dosage. In certain embodiments, the atomizer is connected by one or more electrical connections to a power supply.

In certain embodiments, the medicine delivery controller includes a water reservoir that can be connected to or located adjacent to a bellows that contains a medicine to be aerosolized. In certain embodiments, the water reservoir is located adjacent to or connected to a bottom wall of the bellows, and in proximity to the medicine. In certain embodiments, an ultrasonic atomizer can be placed on a surface of water contained within the water reservoir and adjacent to or connected with the bellows. In certain embodiments, the atomizer is located adjacent to or connected to a bottom wall of the bellows, and in proximity to the medicine. In certain embodiments, the ultrasonic atomizer can be submerged in the water. Such embodiments can provide benefits of translation of sonication or vibrations from the water to the bellows, and provide increased heat capacity.

In certain embodiments, the air flow system includes at least one air tube, at least one air pipe, at least one air path, or a combination thereof. In certain embodiments, the actuator is configured to connect to at least one pressure source. In certain embodiments, the at least one pressure source optionally includes an air pump, an air tank, an air tube, an air line, or a combination thereof. In certain embodiments, the air flow system can include corrugated plastic tubing, a CPAP hose, or a rubber hose.

In certain embodiments, the medicine delivery device further includes a subject interface configured to connect to the air flow system, wherein the subject interface includes a nasal cannula, a face mask, a breathing tube, a medicine port, or a combination thereof. In certain embodiments, the airflow system can be connected to the subject interface using rubber or plastic connectors. Such embodiments including a subject interface in the medicine delivery device can have a benefit of avoiding the necessity of purchasing or supplying a separate subject interface for use of the device. Such embodiments can also provide a benefit of greater utility in use of the device for single patient use.

In certain embodiments, the pressure source can be a pressure source that is incorporated into a positive pressure breathing assistance system. In certain embodiments, the pressure source can be an externally applied pressure source. In certain embodiments, the pressure source can be a separate pressure source included in the medicine delivery device. In certain embodiments, a pressure source included in the medicine delivery device can include an air pump. In certain embodiments, the air pump includes a hand pump. In certain embodiments, the pressure source can be a combination of that of a positive pressure breathing assistance system and a pressure source included in the medicine delivery device, or a combination of an externally applied pressure source and a pressure source included in the medicine delivery device.

In some embodiments, the pressure source, such as an air pump, can be connected to a programmable control module. Embodiments wherein a separate pressure source is included in the medicine delivery device can provide a benefit of being separately controllable from the pressure source of the positive pressure breathing assistance system, thus adding to the versatility and utility of the device. Embodiments allowing direct control of the pressure source by the programmable control module of the medicine delivery device can also provide a benefit of greater accuracy of aerosol medicine delivery during an inhalation by the subject.

In certain embodiments, the programmable control module includes a microcontroller and a programmer interface. In certain embodiments, the microcontroller is directly programmable via the programmer interface. In some embodiments, the programmable control module includes a microcontroller and a wireless transmitter, a wireless receiver, or a combination thereof; or both. Such embodiments can provide benefits of allowing the microcontroller to be remotely programmable, and increasing safety by reducing the requirement for close contact between a patient and a healthcare provider. The microcontroller in various embodiments can allow a healthcare provider to program the microcontroller to deliver desired volumes and rates of delivery of air, oxygen, and aerosol medicine, according to the prescribed treatment for the subject. For example, the microcontroller can be programmed to deliver a prescribed volume of air or oxygen per subject inhalation, or a prescribed amount of aerosol drug per subject inhalation, or a delivery of a prescribed amount of aerosol drug at a prescribed rate over a determined number of breathing actions, such as a dose delivery of drug after a determined number of subject inhalations. In certain embodiments, the delivery of air or oxygen is separately controllable by the microcontroller from the delivery of aerosol drug. The ability to separately program and control the air/oxygen delivery and aerosol drug delivery allows the healthcare provider to prevent the automatic delivery of aerosol drug with every inhalation, thus controlling the frequency of drug delivery and allowing the correct dosage to be supplied at the desired rate of delivery to the patient. In embodiments wherein the programmable control module controls the actuator, the aerosol medicine dispenser, or a combination thereof, the microcontroller can be programmed to sense inhalations and exhalations of a patient, and to control the actuator and the aerosol medicine dispenser to deliver an aerosol medicine dose timed with a patient inhalation.

In certain embodiments, the programmable control module comprises machine-readable code configured to: collect sensor signal data from the at least one non-invasive sensor over a measurement time period; extract respiratory data from the sensor signal data by applying a bandpass filter; determine an inhalation period and exhalation period of the subject; and actuate a treatment during the inhalation period by sending an actuator signal from the programmable control module to the actuator or the aerosol medicine dispenser. When operating the medicine delivery devices of various embodiments, the at least one noninvasive sensor sends a sensor signal to the programmable control module; the programmable control module then controls the actuator, the aerosol medicine dispenser, or a combination thereof, to deliver an aerosol medicine dispensed from the aerosol medicine dispenser to the subject through the air flow system. In an aspect, the device can be configured to deliver an aerosol medicine during an inhalation period by the subject. Embodiments of a machine-readable code can include embodiments of an algorithm herein. Such embodiments can have a benefit of improving the accuracy and efficiency of delivery of a dose of aerosol medicine to the subject, by timing the medicine delivery to an onset of inhalation or during an inhalation by the subject. By targeting delivery of the medicine only during inhalation, the devices of various embodiments can result in more effective treatment of patients with aerosol medicines, thus improving patient outcomes. Such aspects that increase the efficiency of treatment can lead to a beneficial result of reducing the duration of patient hospital stays. Such embodiments can have a benefit of reducing the amount of an aerosol medicine that is required to effectively treat a patient, thus helping to avoid waste and to make more effective use of drugs that may be in short supply, as well as reducing costs, particularly considering the high cost of some drugs used to treat respiratory diseases.

In certain embodiments, the at least one non-invasive sensor comprises an electrocardiogram (ECG) sensor and one or more ECG leads. Such embodiments can provide a benefit of a sensor that is comfortable to wear by the subject for an extended period of time. Use of a non-invasive sensor such as an ECG sensor contrasts to wearable sensors that are available to monitor respiratory rate directly, which are invasive or uncomfortable to wear. In some embodiments, the at least one non-invasive sensor includes a pulse oximeter. Such embodiments can provide a benefit of greater versatility for embodiments of medicine delivery devices for use with different subjects and in a variety of healthcare situations.

In certain embodiments, the medicine delivery device further includes at least one electrical connection, wherein the at least one electrical connection connects the programmable control module to the at least one non-invasive sensor, the programmable control module to the actuator, the programmable control module to the aerosol medicine dispenser, or a combination thereof. The at least one electrical connection can include one or more ECG leads in certain embodiments. In certain embodiments, the at least one electrical connection includes one or more electric wires or cables; in such embodiments, the one or more electric wires or cables can include a sheath material including an electrically insulating material, a rubber material, a flame-retardant material, or a plastic material. In certain embodiments, the at least one electrical connection includes one or more reversible connectors located at a distal end of one or more electric wires, or in line with one or more electric wires. In certain embodiments, a reversible connector can include a USB plug, a cable jack, a coaxial power connector, a banana connector, a plug and socket connector, and a waterproof connector. Such embodiments including one or more reversible electric wire connections can provide benefits of convenience and versatility in connecting and disconnecting various elements of the medicine delivery device according to need. Such embodiments can also provide a benefit of decreasing the risk of the spread of infection by reducing the amount of handling of parts of the medicine delivery device that may become contaminated as a result of such handling.

In certain embodiments, at least one of the programmable control module, the at least one non-invasive sensor, the actuator, and the aerosol medicine dispenser comprises a wireless transmitter, a wireless receiver, or a combination thereof. Such embodiments can provide a benefit of avoiding the use of wires in healthcare situations where the use of wired electrical connections might present a disadvantage, such as with ambulatory subjects or infants. Such embodiments can provide benefits of versatility in the types of electrical connections between the various elements of the medicine delivery device, thus expanding the versatility of the device for use with a variety of different subjects and in various healthcare settings. Such embodiments can also have a benefit of mitigating the risk of the spread of infection by reducing the amount of handling of the device that is required during the course of treatment. Various embodiments of a medicine delivery device can provide a benefit of increasing the versatility of the device for use with a variety of different subjects and in various healthcare settings.

In various embodiments, the medicine delivery device is configured to use a power source. In certain embodiments, the power source includes an external power source, and the medicine delivery device includes at least one electrical connection configured to connect to the external power source. In some embodiments, the medicine delivery device includes at least one electrical connection that connects the programmable control module to an external power source. In an embodiment, the electrical connection can plug into an electrical wall outlet or other external power source. In some embodiments, the medicine delivery device includes an internal power source that is included in the medicine delivery device. In some embodiments, the medicine delivery device can use a combination of an external power source and an internal power source. In certain embodiments, the internal power source can include one or more batteries, or a battery pack. In certain embodiments, one or more batteries or a battery pack can be included in the programmable control module. In such embodiments, the one or more batteries or battery pack can be replaceable, or nonreplaceable. Embodiments wherein the medicine delivery device includes an internal power source can provide benefits of versatility for use of the device in healthcare settings, as well as greater utility of the device for single patient use.

In embodiments of a medicine delivery device herein, various components of the devices can be mostly, if not entirely, formed from a lightweight plastic material, or a cover or housing for various components can be formed from a lightweight plastic material. Such components can include, without limitation, the aerosol medicine dispenser, the air flow system, the actuator, the breath sensor, and the programmable control module. Such embodiments can provide benefits of low cost, lightweight, and compact devices that are simple to transport and store. Yet another benefit can be a utility of the devices for single patient use, so that a device can be used for a single patient and then entirely disposed of, without the need to clean or reuse any parts of the device.

### Embodiments of Methods of Delivering an Aerosol Medicine

Embodiments of methods of delivering an aerosol medicine to a subject in need thereof are disclosed herein. In an embodiment, such a method includes: providing a medicine delivery device, wherein the medicine delivery device comprises an aerosol medicine dispenser connected by an air flow system to an actuator, and a programmable control module configured to control the actuator and the aerosol medicine dispenser; providing at least one non-invasive sensor; attaching the at least one non-invasive sensor to at least one body surface of the subject and configuring the at least one non-invasive sensor to send a sensor signal to the programmable control module; collecting sensor signal data from the at least one non-invasive sensor over a measurement time period; extracting respiratory data from the sensor signal data by applying a bandpass filter; determining an inhalation period and an exhalation period of the subject; and actuating a treatment during the inhalation period by sending an actuator signal from the programmable control module to the actuator or the aerosol medicine dispenser, wherein the actuator or aerosol medicine dispenser is connected by a breathing apparatus connected to the respiratory system of the subject.

Various embodiments of a medicine delivery device herein can provide benefits that can also provide benefits for the use of such devices in embodiments of methods of delivering an aerosol medicine herein. Such benefits can include the accurate and efficient delivery of an aerosol medicine to a subject only during an inhalation phase of breathing, thus helping to improve treatment outcomes, as well as avoiding wastage of expensive medicines; the use of medicine delivery devices having a suitability and versatility for nearly any patient in any healthcare setting, thus helping to reduce recovery times and improve patient outcomes; and the use of medicine delivery devices that can mitigate the risk of the spread of infection by reducing the amount of handling of the device and patient contact required during the course of treatment.

In certain embodiments, the method further includes programming the programmable control module to dispense an amount of medicine for a treatment frequency during a treatment duration. In certain embodiments, the method includes programming the programmable control module to dispense an amount of medicine once per a number of inhalation periods. Embodiments of medicine delivery devices herein can provide an ability to program a delivery of air or oxygen that is separately controllable by the microcontroller from the delivery of an aerosol drug. The ability to separately program and control the air/oxygen delivery and aerosol drug delivery allows the healthcare provider to prevent the automatic delivery of aerosol drug with every inhalation, thus controlling the frequency of drug delivery and allowing the correct dosage to be supplied at the desired rate of delivery to the patient. Embodiments of a medicine delivery device wherein the programmable control module controls the actuator, the aerosol medicine dispenser, or a combination thereof, can provide a benefit of an ability of a healthcare provider to program the control module to sense inhalations and exhalations of a patient, and to control the actuator and the aerosol medicine dispenser to deliver an aerosol medicine dose timed with a patient inhalation.

In some embodiments, the actuator includes a pressure valve, and the method further includes connecting the pressure valve to a pressure source. In certain embodiments, the method further includes flowing a treatment volume of medicine from the aerosol medicine dispenser into the air flow system during an inhalation period. In some embodiments, the method optionally includes closing the pressure valve during an exhalation period.

In certain embodiments, the at least one non-invasive sensor includes an electrocardiogram (ECG) sensor, wherein the method includes attaching the least one non-invasive sensor comprises attaching at least one ECG lead to the body surface of the subject, and the sensor signal data comprises ECG signals. In certain embodiments, the method includes attaching at least two ECG leads to the body surface of the subject, wherein the body surface includes a chest surface, an arm surface, a leg surface, or a combination thereof; and the sensor signal data comprises ECG signals collected from the at least two ECG leads.

In certain embodiments, the at least one non-invasive sensor comprises a pulse oximeter sensor, and the body surface includes a finger surface, a toe surface, an ear surface, or a combination thereof; and wherein the sensor signal data includes oxygen saturation level data.

### EXAMPLES

### Example 1: Generating a Respiratory Pattern from ECG Derived Respiratory Data

ECG data collection from a subject was set up using three electrodes with leads attached to the left arm, right arm, and left leg of the subject. A Sparkfun AD8232 heart rate monitor and an Arduino ECG collection circuit were used to collect the ECG data and convert it into a digital signal.

ECG data collected for a time period of 30 seconds were exported. For the ECG data collection, hardware inputs, outputs, and a communication protocol for Arduino were set up. ECG lead connections were checked using Failsafe. Collected ECG data were instantly plotted in Arduino, or serially outputted, manually copied to .CSV, and visualized in MATLAB. ECG data will be visualized later using Python on a Raspberry Pi.

### Example 2: Extracting a Breathing Signal from Pulse Oximeter Data

Oxygen saturation in the blood (SpO2) is known to changes with inhalation and exhalation. Data from a pulse oximeter were collected from a subject for a time period of 30 seconds and exported, to investigate the circuitry and data collection. A pulse oximeter circuit was constructed using a transimpedance op-amp, an instrumentation amplifier, and a MASIMO pulse oximeter. Red versus infrared (IR) absorbance of the blood based on light shone through tissue, such as a finger, were measured to determine oxygen saturation. Pulsatile transmittance from blue (IR) and red LEDs were observed over the data collection time period. Scoring the collected data indicated that pulse oximeter readings presented a viable option for extracting a respiratory signal.

### Example 3: Extracting a Breathing Signal from Neonatal ECG Data

Pseudocode was written to filter ECG signals and detect inhalation based on neonatal ECG data from Physionet, and output time stamps of when breaths were detected for at least 5 breaths. The pseudo code was written to perform the following steps:

1. Filter signal: bandpass filter with cutoffs at 0.33 Hz and 1.00 Hz
2. Calculate derivative of filtered ECG data: (current-previous data point)/time difference
3. Check if trough: If (derivative at current point)>0 && (derivative at previous point)<0
4. Store current time in array: StartInhaleTimes=time (current point)

### Example 4: Extracting a Breathing Signal from Neonatal ECG Data

In order to filter ECG signals with code, code was written in order to filter ECG sensor signals, based on neonatal ECG data from Physionet. In MATLAB: ECGfilt=bandpass(ecgdata, [lowcut, highcut], fs); lowcut=0.33 Hz; highcut=1 Hz; fs (sampling frequency, given on Physionet)=250 Hz. A representative graph comparing original ECG signals and filtered ECG signals representing extracted respiratory data is shown in FIG. 2.

### Example 5: Extracting a Breathing Signal from Neonatal ECG Data

In order to detect inhalation with code, code was written to detect inhalation based on neonatal ECG data from Physionet and output time stamp of when breaths were detected for at least 5 breaths. The code was written to perform the following steps:

1. Calculate derivative of filtered ECG data: (current-previous data point)/time difference
2. Check if trough: If (derivative at current point)>0 && (derivative at previous point)<0.
3. Store current time in array: StartInhaleTimes=time(current point)

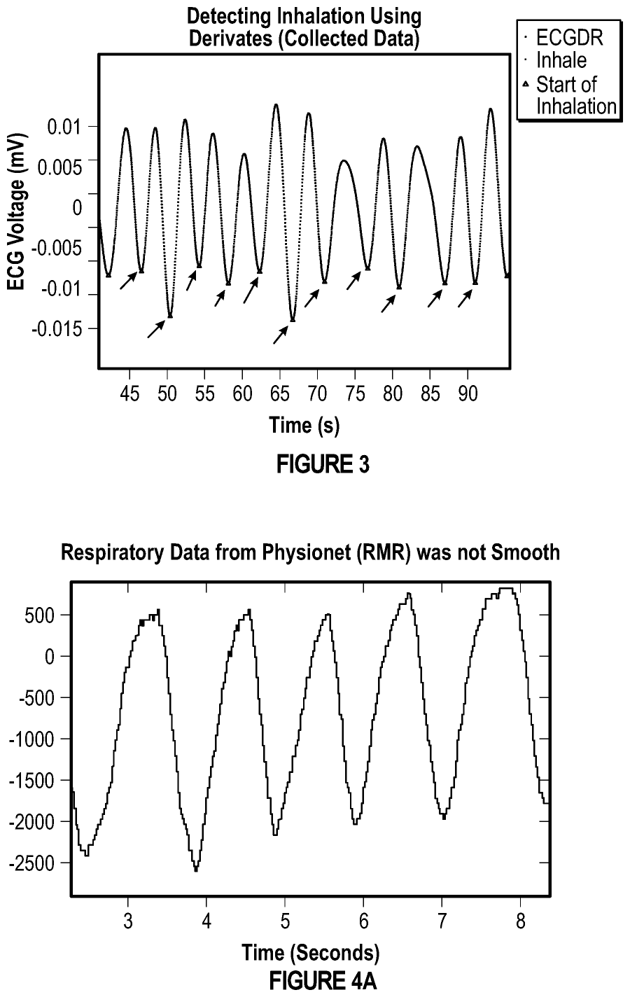

The ECG-derived respiratory signals as ECG voltage (mV) were plotted over time (seconds) in order to detect inhalation points. A representative plot is shown in FIG. 3. In FIG. 3, time points representing the start of inhalations are shown by open triangles and arrows indicating trough inflection points in the ECGDR derivative curve. Inhalation periods are shown as squares from the inhalation start time to the next peak time in the curve.

### Example 6: Extracting a Breathing Signal from Neonatal ECG Data

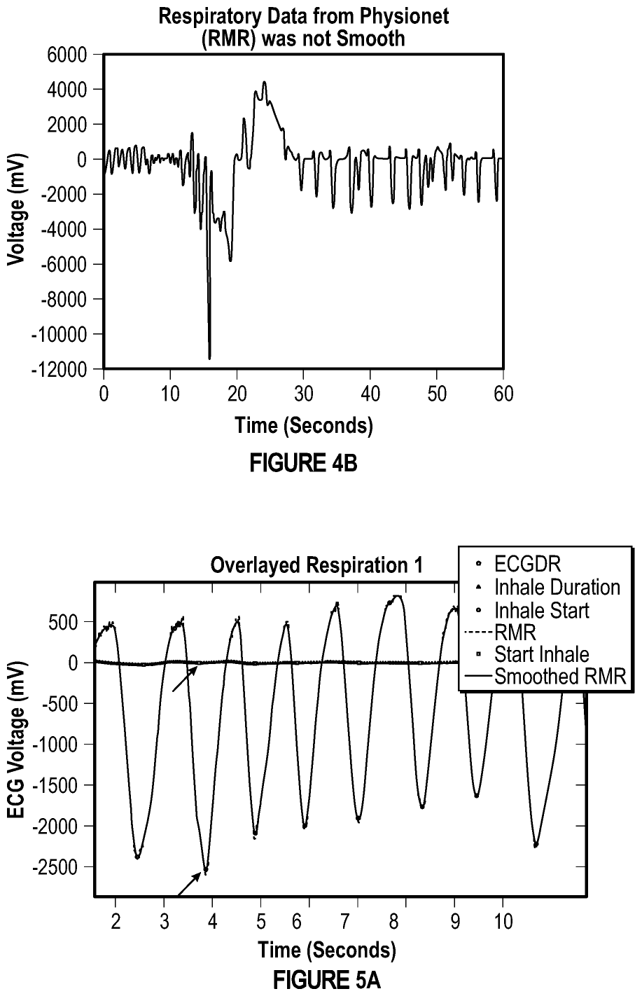

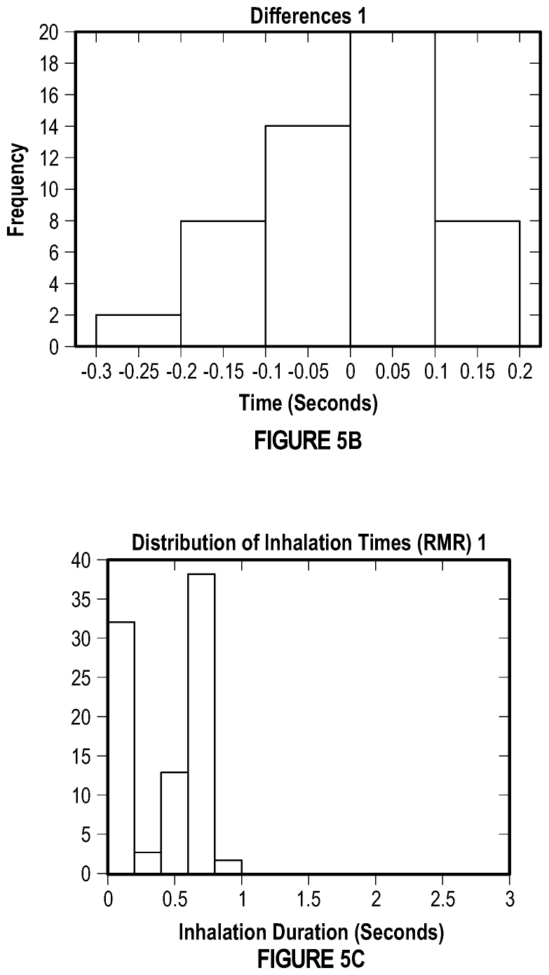

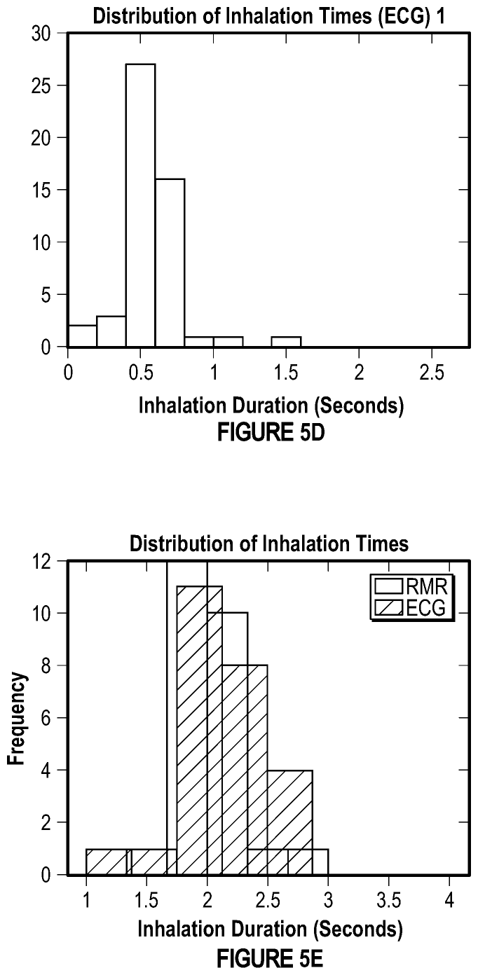

Code was written to compare respiratory signals derived from ECG data from Physionet (ECGDR) to corresponding infant respiratory monitor rate data (RMR) measured using a respiratory monitor, from Physionet. The statistical differences between the respiratory signals (pairwise comparison) was calculated. The respiratory data from Physionet (RMR) was not smooth, as depicted in FIG. 4A and FIG. 4B. The rough RMR data was smoothed using a moving average and plotted in an overlay graph with ECGDR data as ECG voltage (mV) over time, as shown in FIG. 5A for “Infant 1”. In FIG. 5A, the RMR data is shown as the dotted line curve and the smoothed RMR data is shown as the solid line curve, with time points representing the start of inhalations shown by open circles and an arrow indicating a trough inflection point in the RMR curve. Corresponding open circles indicating inhale start times as measured by ECGDR are shown in the ECGDR curve represented by open pentagons. Inhalation periods as measured by ECGDR are shown as open triangle portions in the ECGDR curve. Corresponding inhalation durations as measured by RMR data are shown from the inhalation start time to the next peak time in the RMR curve.

The frequencies of differences in inhalation start times between the RMR and the ECGDR calculations for “Infant 1” are shown in the histogram in FIG. 5B. The mean differences between inhalation start times and standard deviations for Infant 1 and Infants 2, 3, and 4 are shown in Table 1.

TABLE 1

| Infant | Mean(s) | St.Dev.(s) |
|---|---|---|
| 1 | −0.0019 | 0.10 |
| 2 | 0.20 | 0.40 |
| 3 | 0.11 | .40 |
| 4 | −0.12 | 0.35 |

To analyze the statistical differences between inhalation durations between ECGDR and RMR data, the following was performed:

2-sample t-test: population standard deviation (s.d.) unknown

Assume: populations of RMR inhalation duration and ECG inhalation duration are homogeneous
Populations are normally distributed (all breaths are the same duration)
Values are independent
   Hypothesis: μ_{RMR}≠μ_{ECG}
   Alternative Hypothesis: μ_{RMR}≠μ_{ECG}
   For RMR, μ_{RMR}=0.4142 s, σ_{RMR}=0.0891 s², n=51
   For ECG, μ_{ECG}=0.5803 s, σ_{ECG}=0.0379 s², n=88
   t=15.266, p=1.29E-30
   Therefore, there is statistical evidence μ_{RMR}≠μ_{ECG}
   Results for “Infant 1” data are shown in FIG. 5C and FIG. 5D, respectively. In the histograms, distributions of inhalation times for RMR and ECG for Infant 1 are shown for various times of inhalation duration (seconds).

Statistical differences between inhalation times as calculated using RMR versus ECG data is further illustrated in FIG. 5E. In the histogram, distributions of inhalation times for RMR and ECG data are shown overlaid for various times of inhalation duration (seconds). For FIG. 5E:

μ_{RMR}=2.0084 s, σ_{RMR}=0.0827 s², n=25
μ_{ECG}=2.1591 s, σ_{ECG}=0.1228 s², n=25
t=1.662, p=0.103
Since p>α with α=0.05, there is no statistical evidence the means are unequal, therefor μ_{RMR}=μ_{ECG}.

### Example 7. Extracting a Breathing Signal from ECG Data Compared to Breathing Signals Measured in Real Time

ECG data and data corresponding to the duration of inhalation of a team member were simultaneously collected, so that the inhalation time period of the ECG signals could be determined. This will be used to determine the accuracy of respiratory signals derived from the ECG data. An ECG and inhalation collection circuit was constructed for simultaneous ECG collection and button collection. While ECG data was collected, the team member pressed a button to signal times of inhalation and exhalation start times. For the data collection, the following data treatment steps were carried out:
1. Initialized data storage length and variables for ECG traces and button pressing
2. Begin timer
3. Collect data: collect ECG traces and collect and normalize button signal
4. End timer and calculate sampling frequency
5. Save data into .csv and visualize in MATLAB

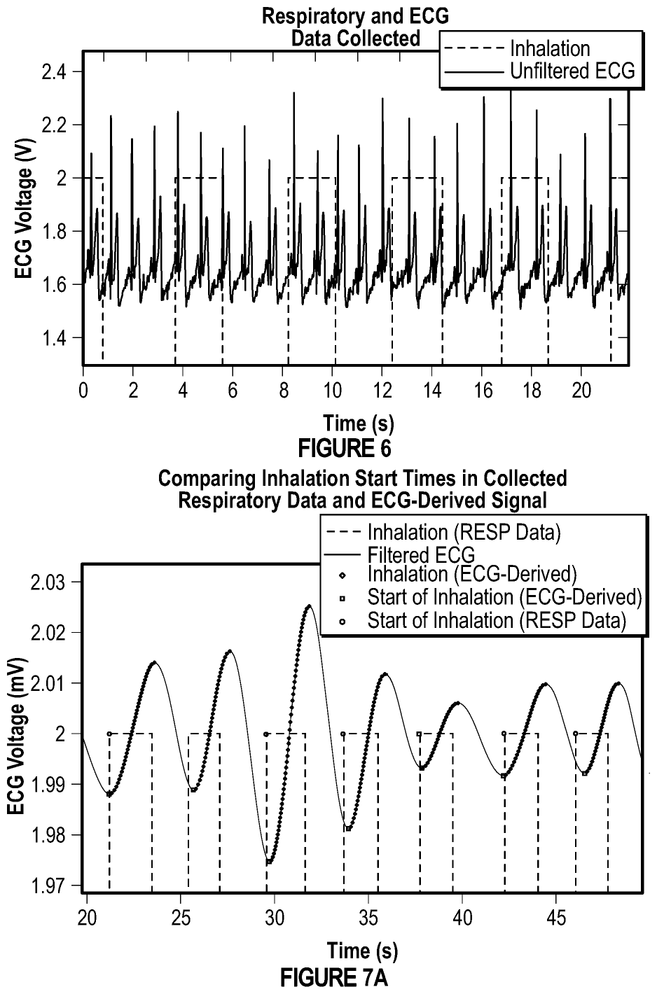

Simultaneous collection of ECG data and data corresponding to the duration of inhalation of a team member are shown in FIG. 6. In the graph, unfiltered ECG data and corresponding inhalation data (boxed lines) are shown superimposed.

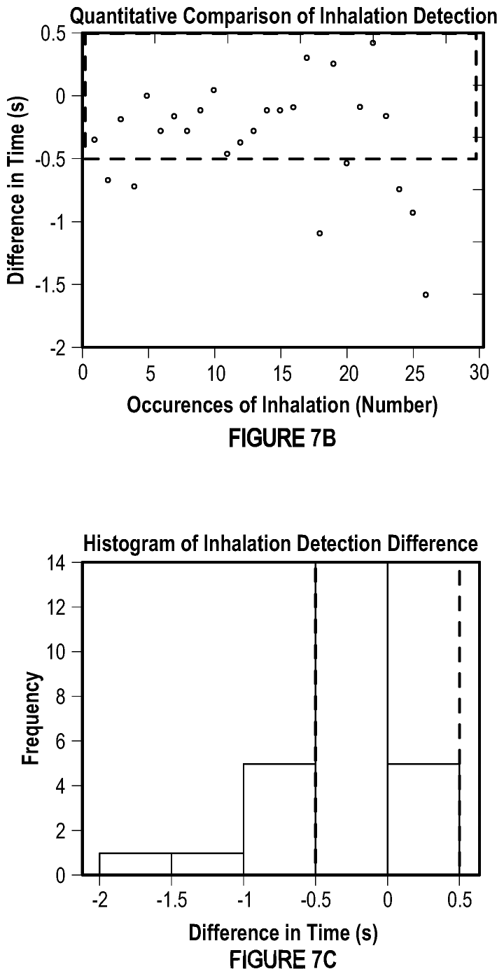

A statistical comparison of the respiratory signal derived from ECG data and corresponding respiratory data is shown in FIG. 7A. In FIG. 7A, filtered ECGDR respiratory curve data (solid line) is shown with open boxes indicating ECG inhalation start times and open diamonds showing ECG-derived inhalation periods. Inhalation (respiratory data) is shown (dashed lines), with open circles indicating inhalation start times as measured by button pressing.

FIG. 7B shows a dot plot with a quantitative comparison of inhalation detection from the data shown in FIG. 7A. The differences in time(s) between inhalation start times between ECG data and corresponding respiratory data are shown plotted against the number of occurrences of inhalation. The dashed line box indicates the data as shown in the histogram in FIG. 7C. FIG. 7C shows a histogram of the frequency of differences in inhalation detection times between the ECG data and corresponding respiratory data. The dashed line box shows the data reflected by the corresponding box in FIG. 7B. For the data, the average difference was −0.32 seconds, and the standard deviation was 0.44 seconds.

### Example 8: Extracting a Breathing Signal to Detect Inhalation in Real Time

To detect inhalation from ECG sensor data, code was written to detect inhalation using the sensor data in real time (the initial criteria for defining real time was begin able to detect the start of inhalation within maximum 1 minute after it occurs in the subject.) In order to perform real-time ECG data filtering, code was written according to the flow chart shown in FIG. 8. With the use of the code, inhalation can be detected from ECG sensor data within one minute:

with a delay of 81 milliseconds.

An output time stamp of breaths was detected for at least 5 breaths. The following while loop was utilized, in writing code to detect inhalation based on neonatal ECG data from Physionet:

```
MATLAB:
while n < length(ecgFiltered) − 1
  if (ecgDeriv(n) > 0) && (ecgDeriv(n−1) < 0) % look at where
function is increasing after a previous decrease (a trough)
    ecgStartInhalePts(end+1) = ecgFiltered(n); % add to subset of
  points and times of start of inhalation
    ecgStartInhaleTimes(end+1) = ecgTimes(n) % outputs the times
end
  if (ecgDeriv(n) > 0) % look at where function is increasing (entire
  duration of inhalation)
    ecgInhalePts(end+1) = ecgFiltered(n); % add to subset
    ecgInhaleTimes(end+1) = ecgTimes(n);
end
n = n+1;
end
```

Pseudocode was written to filter ECG signals and detect inhalation. The following steps were used:

1. Filter ECG Signal: bandpass filter with cutoffs of 20 and 60 breaths/minute; 20-6-breaths per minute (BPM) is 0.33 Hz to 1.0 Hz.
2. Detect inhalation:
a. Calculate derivatives of filtered ECG signals using (current−previous data point)/time difference
b. If the derivative at current point>0 and the derivative at a previous data point<0
   Start of inhalation
   Store time at this current data point into an array (StartInhaleTimes)
c. Plot original ECG signal, filtered ECG signal, and mark points where start of inhalation was detected
d. Display the array of inhalation start times (StartInhaleTimes)

Code was written to detect inhalation from ECG sensor data in real time, using the sensor's data within 1 minute. A “while loop” such as used in this code is depicted in FIG. 9.

### Example 9: Medicine Delivery Device

A medicine delivery device will be constructed for delivering an aerosol medicine to an infant subject. The medicine delivery device will include: (a) Drug storage: An aerosol medicine dispenser receptacle to hold a dose of liquid surfactant, (b) Aerosol generation: A high-frequency vibrating piezoelectric that converts liquid to aerosol, (c) Physical connections: A connection to the infant's cannula interface and (d) Aerosol propulsion: A device that provides of a bolus of air to push the aerosol through the cannula interface upon inhalation. The propulsion/control/actuation can be provided by a pressure valve or the mechanical compression of a flexible bellows. The programmable control module would then control the actuation component is (either a valve or a motor/other type of actuator).

Actuation can also be triggered by the programmable control module by turning the aerosolizer unit on and off, as opposed to the pressure valve.

What is claimed is:

1. A method of treating a respiratory system of a subject comprising:
providing at least one non-invasive sensor;
attaching the least one non-invasive sensor to at least one body surface of the subject and configuring the at least one non-invasive sensor to send a sensor signal to a controller;
collecting sensor signal data from the at least one non-invasive sensor over a measurement time period;
extracting respiratory data from the sensor signal data by applying a bandpass filter; determining an inhalation period and exhalation period of the subject; and
actuating a treatment during the inhalation period by sending an actuator signal from the controller to an air pump or a medicine delivery device, wherein the pump or medicine delivery device is connected by a breathing apparatus connected to the respiratory system of the subject.

2. The method of claim 1, further comprising generating a respiratory pattern of the subject by applying an algorithm to the extracted respiratory data, wherein applying the algorithm comprises calculating derivatives of the extracted respiratory data as a function of time to form a derivative curve, and optionally, wherein an inflection point of the extracted respiratory data corresponding to a change in sign in the derivative curve corresponds to a time of onset of an inhalation period or a time of onset of an exhalation period.

3. The method of claim 1, wherein the subject is a human, an infant, an unconscious patient, a patient receiving a mechanically assisted breathing treatment, a ventilated patient, a cat, a dog, a horse, or a mammal.

4. The method of claim 1, wherein the at least one non-invasive sensor comprises an electrocardiogram (ECG) sensor, wherein attaching the least one non-invasive sensor comprises attaching at least one ECG lead to the body surface of the subject, and the sensor signal data comprises ECG signals.

5. The method of claim 4, comprising attaching at least two ECG leads to the body surface of the subject, wherein the body surface includes a chest surface, an arm surface, a leg surface, or a combination thereof; and the sensor signal data comprises ECG signals collected from the at least two ECG leads.

6. The method of claim 1, wherein the measurement time period is from about 10 seconds to about 2 minutes; or wherein the measurement time period comprises from about 3 to about 120 repeated inhalation periods or exhalation periods of the subject.

7. The method of claim 1, wherein applying the bandpass filter includes applying a lower cutoff inhalation or exhalation frequency of about 0.33 Hz and a higher cutoff inhalation or exhalation frequency of about 1 Hz; or wherein applying the bandpass filter includes applying a sensor signal data sampling frequency of about 250 Hz.

8. The method of claim 1, wherein the at least one non-invasive sensor comprises a pulse oximeter sensor, and the body surface includes a finger surface, a toe surface, an ear surface, or a combination thereof; and wherein the sensor signal data includes oxygen saturation level data.

9. A method of delivering an aerosol medicine to a subject in need thereof comprising:
providing a medicine delivery device, wherein the medicine delivery device comprises an aerosol medicine dispenser connected by an air flow system to an actuator, and a programmable control module configured to control the actuator and the aerosol medicine dispenser;
providing at least one non-invasive sensor;
attaching the at least one non-invasive sensor to at least one body surface of the subject and configuring the at least one non-invasive sensor to send a sensor signal to the programmable control module;
collecting sensor signal data from the at least one non-invasive sensor over a measurement time period;
extracting respiratory data from the sensor signal data by applying a bandpass filter;
determining an inhalation period and an exhalation period of the subject; and
actuating a treatment during the inhalation period by sending an actuator signal from the programmable control module to the actuator or the aerosol medicine dispenser, wherein the actuator or aerosol medicine dispenser is connected by a breathing apparatus connected to the respiratory system of the subject.

10. The method of claim 9, further comprising programming the programmable control module to dispense an amount of medicine for a treatment frequency during a treatment duration; or programming the programmable control module to dispense an amount of medicine once per a number of inhalation periods.

11. The method of claim 9, wherein the actuator comprises a pressure valve, and further comprising connecting the pressure valve to a pressure source; or further comprising flowing a treatment volume of medicine from the aerosol medicine dispenser into the air flow system during an inhalation period; and optionally, closing the pressure valve during an exhalation period.

12. The method of claim 9, wherein the at least one non-invasive sensor comprises an electrocardiogram (ECG) sensor, wherein attaching the least one non-invasive sensor comprises attaching at least one ECG lead to the body surface of the subject, and the sensor signal data comprises ECG signals.

13. The method of claim 12, comprising attaching at least two ECG leads to the body surface of the subject, wherein the body surface includes a chest surface, an arm surface, a leg surface, or a combination thereof; and the sensor signal data comprises ECG signals collected from the at least two ECG leads.

14. The method of claim 9, wherein the at least one non-invasive sensor comprises a pulse oximeter sensor, and the body surface includes a finger surface, a toe surface, an ear surface, or a combination thereof; and wherein the sensor signal data includes oxygen saturation level data.

15. A medicine delivery device comprising:
an aerosol medicine dispenser connected by an air flow system to an actuator;
at least one non-invasive sensor configured to attach to at least one body surface of a subject; and
a programmable control module configured to receive sensor signals from the at least one non-invasive sensor and configured to control the actuator and the aerosol medicine dispenser.

16. The medicine delivery device of claim 15, wherein the programmable control module comprises machine-readable code configured to:
collect sensor signal data from the at least one non-invasive sensor over a measurement time period;
extract respiratory data from the sensor signal data by applying a bandpass filter;
determine an inhalation period and exhalation period of the subject; and
actuate a treatment during the inhalation period by sending an actuator signal from the programmable control module to the actuator or the aerosol medicine dispenser.

17. The medicine delivery device of claim 15, wherein the at least one non-invasive sensor comprises an electrocardiogram (ECG) sensor and one or more ECG leads; or the at least one non-invasive sensor comprises a pulse oximeter; or further comprising a subject interface configured to connect to the air flow system, wherein the subject interface includes a nasal cannula, a face mask, a breathing tube, a medicine port, or a combination thereof.

18. The medicine delivery device of claim 15, wherein the aerosol medicine dispenser comprises a medicine delivery controller connected to a dispensing opening of a medicine reservoir,
wherein the medicine delivery controller is connected to the air flow system, and
wherein the medicine delivery controller comprises a nebulizer, an aerosolizer, an atomizer, a pressurized metered dose inhaler, a vaporizer, a fan, a hopper, a dry powder inhaler, a diffuser, a vibrating piezoelectric aerosolizer, or a combination thereof; or wherein the actuator comprises a pressure valve, a flexible bellows, a motor, a hand pump, a solenoid valve, an air flow valve, or a combination thereof.

19. The medicine delivery device of claim 15, further comprising at least one electrical connection, wherein the at least one electrical connection connects the programmable control module to the at least one non-invasive sensor, the programmable control module to the actuator, the programmable control module to the aerosol medicine dispenser, or a combination thereof; or
wherein at least one of the programmable control module, the at least one non-invasive sensor, the actuator, and the aerosol medicine dispenser comprises a wireless transmitter, a wireless receiver, or a combination thereof.

20. The medicine delivery device of claim 15, wherein the air flow system comprises at least one air tube, at least one air pipe, at least one air path, or a combination thereof; or wherein the actuator is configured to connect to at least one pressure source, and optionally, the at least one pressure source comprises an air pump, an air tank, an air tube, an air line, or a combination thereof.
